(19) United States
(12) Patent Application Publication
(10) Pub. No.: US 2006/0195048 A1
(43) Pub. Date: Aug. 31, 2006

(54) Method and apparatus for determining the angle between the femur and the tibia

(75) Inventors: Francois Leitner, Uriage (FR); Yann Laugier, Jieres (FR)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/372,247

(22) Filed: Mar. 8, 2006

Related U.S. Application Data
(63) Continuation of application No. PCT/EP03/10215, filed on Sep. 13, 2003.

(51) Int. Cl. A61B 5/103

(52) U.S. Cl. 600/587

(57) ABSTRACT

In a method for determining the angle between the femur and the tibia in the implantation of a unicondylar knee prosthesis, in order to determine in advance the influence of the implantation position of the implant on the angle between the femur and the tibia, it is proposed that the position of the unaltered joint surface on the tibia and/or the femur be determined, that the position of a bearing surface for a unicondylar implant on the tibia and/or the femur be determined, and that the angle between the femur and the tibia be calculated using these position data and the geometrical data relating to the unicondylar implant.

This application is a continuation of international application number PCT/EP2003/010215 filed on Sep. 13, 2003.

The present disclosure relates to the subject matter disclosed in international application number PCT/EP2003/010215 of Sep. 13, 2003, which is incorporated herein by reference in its entirety and for all purposes.

### BACKGROUND OF THE INVENTION

The invention relates to a method for determining the angle between the femur and the tibia in the implantation of a unicondylar knee prosthesis and to an apparatus for performing this method comprising a navigation system and a data processing unit.

Besides replacing a natural knee joint by a complete knee endoprosthesis, it is desirable, in some cases, to replace the natural knee joint on one side only, i. e., to replace only one of the two condyles of the knee joint with an endoprosthesis, but to leave the other joint surface unaltered. When implanting such unicondylar endoprostheses, a unicondylar implant is placed on the femur and on the tibia, respectively, and these two implants, possibly with a glide component placed therebetween, replace the natural joint surfaces of the femur and the tibia on one side. For implantation of these unicondylar implants, both the tibia and the femur have to be machined. The natural joint surfaces must be removed, and bearing surfaces for these unicondylar implants must be made in the bones. The position of these bearing surfaces must be selected so as to correspond to the dimensions of the unicondylar implants used.

It has now emerged that the position of these bearing surfaces and, consequently, the position of the unicondylar implants affects the angle between the longitudinal axis of the femur and the longitudinal axis of the tibia very sensitively. This applies both with respect to a translational movement of the implant parallel to the longitudinal direction of the femur or the tibia and with respect to a pivotal movement of the implant relative to a plane extending perpendicularly to the longitudinal axis of the tibia or the femur.

The object of the invention is to indicate a method for determining the angle between the femur and the tibia in dependence upon the implantation position of the unicondylar implants so as to find the desired position for the bearing surface on the basis of this determination.

### SUMMARY OF THE INVENTION

This object is accomplished in a method of the kind described at the outset, in accordance with the invention, in that the position of the unaltered joint surface on the tibia and/or the femur is determined, the position of a bearing surface for a unicondylar implant on the tibia and/or the femur is determined, and the angle between the femur and the tibia is calculated using these position data and the geometrical data relating to the unicondylar implant.

The described method may be performed either on the tibia or on the femur or on both bones. It is of fundamental importance that the bearing surface for the unicondylar implant be determined, and on the basis of the position data, i. e., the position in space and the orientation in space, and on the basis of the dimensions of the desired implant, together with the position data relating to the unaltered joint surface, the overall geometry of the knee be calculated.

The angle between the longitudinal axis of the tibia and the longitudinal axis of the femur then also results from this overall geometry. This angle can be influenced by changing the position data relating to the bearing surface, and the operator, therefore, has the possibility of making corrections in the angle.

All these procedures are carried out before the bearing surfaces are made in the bones, so that the operator can vary the position of the bearing surface arbitrarily before machining the bone, so as to achieve optimum adaptation. This also applies with respect to selection of implants with suitable geometrical data. The angle between the femur and the tibia can also be influenced by selecting implants with different geometrical data and correspondingly adapted position of the bearing surface.

It is expedient for the position of the unaltered joint surface to be determined by determining the position of at least one selected point on the unaltered joint surface. It is possible to determine the unaltered joint surface by only one point, for example, the lowest point on the tibia joint surface. It is, however, also possible to detect, for example, the position of several distinctive points on this joint surface, in order to determine the position of the unaltered joint surface.

In particular, the selected point or points on the unaltered joint surface can be detected by palpation.

It is expedient for the selected point or points on the unaltered joint surface to be determined by determining the position of a navigated palpation instrument with which the selected points are approached.

In a particularly preferred embodiment it is provided that the position of the bearing surface is defined by an instrument arranged beside the tibia and/or the femur before preparing the bearing surface. The position of the instrument is preferably determined by a navigation system, so that indirectly by way of the instrument the position of the bearing surface is also determined by the navigation system.

It is expedient to use a saw guide as instrument. This is then simultaneously used to guide a saw with which the bearing surface is made in the bones.

The angle between the femur and the tibia is preferably displayed on a display screen, so that the operator sees immediately what influence is had on the angle between the femur and the tibia by a change in position of the bearing surface and hence of the unicondylar implant. For example, the operator can move the saw guide beside the bone to be worked on until the angle between the femur and the tibia assumes the desired size, and this saw guide can then be fixed in the attained position relative to the bone, so that the position of the bearing surface corresponding to the desired angle between the femur and the tibia is then secured when making the saw cut.

The object underlying the invention is also to so configure a generic apparatus that with it the angle between the femur and the tibia is determinable in dependence upon the implantation position of the unicondylar implant.

This object is accomplished with an apparatus of the kind described at the outset, in accordance with the invention, in that an instrument whose position relative to the position of the unaltered joint surface is determinable by the navigation system is provided for defining the position of a bearing surface for a unicondylar implant on the tibia and/or on the femur, and in that the data processing unit calculates the angle between the femur and the tibia using these position data and the geometrical data relating to the unicondylar implant.

In particular, this apparatus may comprise a navigated palpation instrument for determining the position of the unaltered joint surface.

It is particularly advantageous for the instrument for defining the position of a bearing surface for a unicondylar implant on the tibia and/or on the femur to be a saw guide.

Furthermore, it may be provided that the apparatus comprises a display screen for displaying the angles between the femur and the tibia as calculated by the data processing unit.

The following description of preferred embodiments of the invention serves in conjunction with the drawings to explain the invention in greater detail.

### BRIEF DESCRIPTION OF THE DRAWINGS

FIG. 1 shows a schematic view of an apparatus for determining the angle between the femur and the tibia with a navigation system, a data processing unit and a navigated instrument for defining the bearing surface of a unicondylar implant; and

FIG. 2 shows a schematic view of the proximal tibia end with an unaltered joint surface and a joint surface replaced by a unicondylar implant.

### DETAILED DESCRIPTION OF THE INVENTION

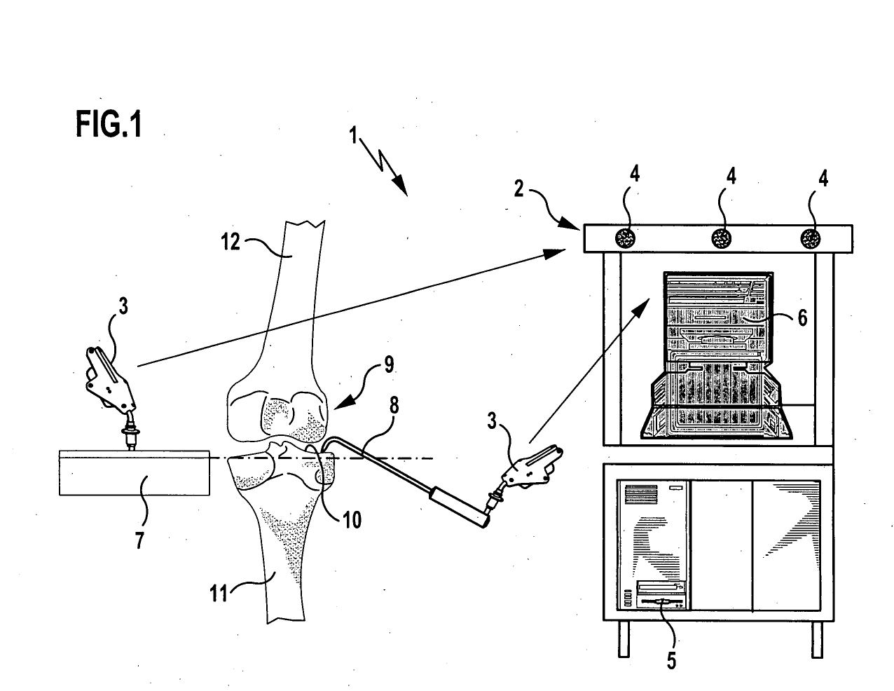

The apparatus 1 shown in FIG. 1 comprises a navigation system 2 of a kind known per se, with which the location of markers 3, i. e., the position and orientation, can be established. These markers 3 can be rigidly attached to instruments and other objects, so that the position of these instruments in space is thereby also detectable.

Such navigation systems 2 operate, for example, with the aid of infrared radiation, which is emitted from several transmitting and receiving devices 4 spaced from one another, is reflected at different locations on the marker 3 and is then received again by the transmitting and receiving devices 4. The thus obtained position data relating to the marker 3 and hence the object to which the marker 3 is attached are fed by the navigation system 2 to a data processing unit 5, with which a display screen 6 is associated.

The described apparatus 1 further comprises a saw guide 7, shown only very schematically in the drawings, which serves to guide an oscillating saw blade of a bone saw in a plane, and a palpation instrument 8 for approaching selected points on bone structures with the tip thereof. The saw guide 7 and the palpation instrument 8 are respectively connected to a marker 3, so that the position of saw guide and palpation instrument is continuously detectable by the navigation system 2.

The described apparatus 1 serves to prepare a knee joint 9 for the implantation of a unicondylar knee endoprosthesis. During this operation, one of the two joint surfaces 10 remains unaltered, while the other joint surface is removed and replaced on both the tibia 11 and the femur 12 by a unicondylar implant 13.

To prepare for the operation, a longitudinal axis is first determined in a manner known per se for both the tibia 11 and the femur 12. This may be achieved by, for example, the center point of the knee joint and the center point of the hip joint or the ankle joint being determined for the two bones. These points are used for defining the longitudinal axes. When determining these, both femur and tibia are provided with further markers, which are not shown in the drawings. These markers then also serve to determine the position of femur and tibia by means of the navigation system.

Geometrical data relating to the unaltered joint surface 10 are recorded with the aid of the palpation instrument 8. For this purpose, the palpation instrument 8 is used to approach either only one selected point or, optionally, several selected points, and the position data relating thereto are stored in the data processing unit 5.

The saw guide 7 is placed beside the bone to be worked on and orientated so that the sawing plane defined by it defines a bearing surface 14 for the implant 13. This bearing surface 14 will, as a rule, lie in the same plane as that in which the saw guide 7 guides the saw blade of a saw. The plane defining the bearing surface 14 then lies beside the saw guide 7. The position data for a certain position of the saw guide and hence for a certain assumed bearing surface 14 are also fed to the data processing unit 5 and stored there.

Finally, the geometrical data relating to the implant 13 used, for example, the height of the implant, are also stored in this data processing unit 5.

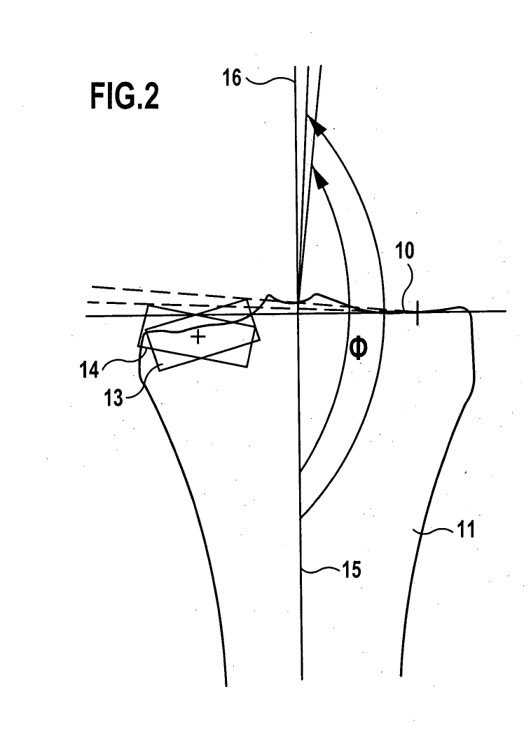

The angle between the longitudinal axes of the tibia and the femur is calculated by the data processing unit 5 from these data stored in the data processing unit 5. This angle depends on the geometrical data relating to the implant and on the position of the implant in the bone. Both an axial displacement in the direction of the longitudinal axis of the bones and a pivotal movement result in a different positioning of the implant relative to the unaltered joint surface 10 and hence in a change in the angle Φ between the longitudinal axis 15 of the femur 12 and the longitudinal axis 16 of the tibia 11. FIG. 2 shows various such angles which may result from the different positioning of the implant 13. It is, of course, also taken into account how the implant is arranged on the respective other bone. Therefore, with respect to the arrangement of the implant on the other bone, it is either based on a certain position, which is assumed, or a position of the bearing surface is assumed in a similar way on both bones and varied by means of saw guide 7 until the desired angular orientation of the longitudinal axes 15 and 16 is obtained.

The data processing unit 5 transmits a diagrammatic image of the knee joint to the display screen 6 and represents on it the angle resulting between the longitudinal axis 15 and the longitudinal axis 16. Optionally, two views rotated through 90° can be presented on the display screen, so that the angular position is visible in different directions. The representation on the display screen may correspond approximately to the illustration in FIG. 2, in which the unaltered joint surface and the position and orientation of the implant 13 corresponding to the assumed position of the bearing surface 14 are shown schematically alongside one another. In addition, the longitudinal axes 15 and 16 and the angle Φ included by these are represented.

If the operator changes the position of the saw guide 7 relative to the bone, this means that the assumed position of the bearing surface 14 is also displaced, and this results directly in a change in the angle Φ between the two longitudinal axes 15 and 16. Consequently, by changing the position of the bearing surface 14, the operator can set the desired orientation of these longitudinal axes and hence determine the bearing surface 14 that has to be made in the bone for a specific implant 13.

1. Method for determining the angle between the femur and the tibia in the implantation of a unicondylar knee prosthesis, wherein the position of the unaltered joint surface on the tibia and/or the femur is determined, the position of a bearing surface for a unicondylar implant on the tibia and/or the femur is determined, and the angle between the femur and the tibia is calculated using these position data and the geometrical data relating to the unicondylar implant.

2. Method in accordance with claim 1, wherein the position of the unaltered joint surface is determined by determining the position of at least one selected point on the unaltered joint surface.

3. Method in accordance with claim 2, wherein the selected point or points of the unaltered joint surface is or are determined by palpation.

4. Method in accordance with claim 2, wherein the selected point or points of the unaltered joint surface is or are determined by determining the position of a navigated palpation instrument.

5. Method in accordance with claim 1, wherein the position of the bearing surface is defined by means of an instrument arranged beside the tibia and/or the femur before preparing the bearing surface.

6. Method in accordance with claim 5, wherein the position of the instrument is determined by means of a navigation system.

7. Method in accordance with claim 5, wherein a saw guide is used as instrument.

8. Method in accordance with claim 6, wherein a saw guide is used as instrument.

9. Method in accordance with claim 1, wherein the angle between the femur and the tibia is displayed on a display screen.

10. Apparatus for determining the angle between the femur and the tibia in the implantation of a unicondylar knee prosthesis, comprising a navigation system and a data processing unit, wherein an instrument whose position relative to the position of the unaltered joint surface is determinable by the navigation system is provided for defining the position of a bearing surface for a unicondylar implant on the tibia and/or on the femur, and the data processing unit calculates the angle (Φ) between the femur and the tibia using these position data and the geometrical data relating to the unicondylar implant.

11. Apparatus in accordance with claim 10, wherein the apparatus comprises a navigated palpation instrument for determining the position of the unaltered joint surface.

12. Apparatus in accordance with claim 10, wherein the instrument for defining the position of a bearing surface for a unicondylar implant on the tibia and/or on the femur is a saw guide.

13. Apparatus in accordance with claim 11, wherein the instrument for defining the position of a bearing surface for a unicondylar implant on the tibia and/or on the femur is a saw guide.

14. Apparatus in accordance with claim 10, wherein the apparatus comprises a display screen for displaying the angles (Φ) between the femur and the tibia as calculated by the data processing unit.
